(19) United States
(12) Patent Application Publication
(10) Pub. No.: US 2008/0008768 A1
(43) Pub. Date: Jan. 10, 2008

(54) Compositions and Methods for Inhibiting Sonic Hedgehog Activity

(76) Inventors: David J. Robbins, Hanover, NH (US); Ziqiang Yuan, Burlington, VT (US); John A. Goetz, Bloomington, IN (US); William J. Scott JR., Cincinnati, OH (US); Sheila M. Bell, Cincinnati, OH (US); Claire M. Schreiner, Cincinnati, OH (US)

Correspondence Address: LICATA & TYRRELL P.C., 66 E. MAIN STREET, MARLTON, NJ, 08053, US

(21) Appl. No.: 11/718,752

(22) PCT Filed: Nov. 9, 2005
(86) PCT No.: PCT/US05/40613

§ 371 Date: Aug. 15, 2007

Related U.S. Application Data
(60) Provisional application No. 60/627,553, filed on Nov. 12, 2004.

(51) Int. Cl. A61K 33/24; A61P 35/00; G01N 33/53

(52) U.S. Cl. 424/654; 435/7.1

(57) ABSTRACT

The present invention relates to the use of sonic hedgehog antagonists in the prevention and treatment of cancer and other hyperproliferative diseases or disorders. Methods for identifying such antagonists are also provided.

### INTRODUCTION

This invention was supported in part by funds from the U.S. government (NIH Grant No. GM 064011). Therefore, the U.S. government may have certain rights in the invention.

### BACKGROUND OF THE INVENTION

Sonic Hedgehog (Shh) has been implicated as the active component in numerous tissues that exhibit organizer and patterning activity. The expression pattern of Shh is consistent with different developmental abnormalities observed in humans with mutations in downstream Shh pathway components. One of these components is also found mutated in the developmental disorder known as Gorlin‘s syndrome. Besides numerous developmental abnormalities, individuals afflicted with this disorder have an inherited predisposition to basal cell carcinoma, rhabdomyosarcoma, as well as to a variety of primitive neuroectodermal tumors. Mutations in this inherited disorder map to the hPTC gene. The loss-of-function mutations found in both hereditary and sporadic cases of basal cell carcinoma implicate hPTC as a tumor suppressor in this common human cancer. Consistent with the role of hPTC as a tumor suppressor, mice heterozygous for Ptc develop a variety of cancers as they age. Other components of the Shh pathway, such as the gene encoding the seven-transmembrane protein hSMO, are also found mutated in sporadic forms of these same malignancies, suggesting they may be involved in human cancer development.

Overexpression of Shh itself has been implicated in a number of more common human tumors, including lung and pancreatic cancer. No mutations of hPTC or SMO were found in these tumors; instead increased production of Shh was implicated in tumor development. In cell lines derived from these tumors, Shh, expressed in an autocrine fashion, activated a Shh-dependent luciferase reporter gene and activated the Shh target gene hPTC. These various tumor cell lines appeared to be dependent upon Shh for their survival, as the addition of the hSMO antagonist cyclopamine, or an inhibitory Shh antibody, led to tumor cell apoptosis (Sheng, et al. (2004) Mol. Cancer 3(1):29; Qualtrough, et al. (2004) Int. J. Cancer 110(6):831-7; Nishimaki, et al. (2004) Biochem. Biophys. Res. Commun. 314(2):313-20; Thayer, et al. (2003) Nature 425(6960):851-6; Tabs and Avci (2004) Eur. J. Dermatol. 14(2):96-102). Shh also appeared to act as a mitogen for these cell lines, as recombinant Shh was sufficient to induce cell division. Similar results were obtained from xenografts of freshly biopsied human tumors. Thus, the Shh pathway may play a crucial role in maintaining tumor cell survival in many common tumors.

### SUMMARY OF THE INVENTION

The present invention is a method for reducing the proliferation of a cancer cell by contacting a cancer cell which is dependent upon Shh activity with an effective amount of an agent which inhibits the activity of Shh thereby reducing the proliferation of the cancer cell. In particular embodiments, the agent is cadmium.

The present invention also relates to a method for preventing or treating cancer by administering to a patient having or at risk of having a cancer which is dependent upon Shh activity with an effective amount of an agent which inhibits the activity of Shh thereby preventing or treating the cancer. In particular embodiments of this method of the invention, the agent is cadmium.

The present invention further relates to a method for identifying an agent which inhibits the activity of Sonic Hedgehog (Shh) in a cell. The method involves contacting a cell expressing Sonic Hedgehog with a test agent and determining whether the test agent inhibits the activity of Sonic Hedgehog in the cell as compared to a cell not contacted with the test agent thereby identifying an agent which inhibits the activity of Shh.

### DETAILED DESCRIPTION OF THE INVENTION

Shh plays a critical role in the development of limb structures. Complete loss of Shh throughout the embryo by conventional gene knockout strategy results in a complete loss of the autopod and loss of the posterior skeletal structure in the zeugopod (Kraus, et al. (2001) Mech. Dev. 100:45-58; Chiang, et al. (2001) Dev. Biol. 236:421-435). Further, right forelimb ectrodactyly is the phenotype of mice lacking a functional allele of Shh coupled with a lox P-conditional limb mesenchyme allele (Lewis, et al. (2001) Cell 105:599-612). Offspring with this genotype consistently miss digits from the posterior aspect of the forelimb. Similarly, postaxial right forelimb ectrtodactyly can be induced by acetazolamide treatment and signaling by the Shh protein is almost totally abolished after acetazolamide exposure as measured by polarizing activity of the mouse embryo zone of polarizing activity (ZPA) (Bell, et al. (1999) Mech. Dev. 88:147-157). However, the expression of Shh is not perturbed by acetazolamide exposure, nor is Patched (Ptch1), the receptor for Shh, whose expression is sensitive to Shh. Like acetazolzmide, cadmium also induces a forelimb phenotype, and does so in a high frequency of embryos at a dosage that is only slightly embryolethal. Therefore, the mechanism by which cadmium induces forelimb ectrtodactyly was determined.

Because agents such as cadmium induce postaxial ectrodactyly that progresses anteriorly as dosage is increased, it was determined whether the teratogenic activity impaired the process responsible for A/P pattern formation in the limb. A/P patterning of the vertebrate limb is under the direction of Shh signaling (McMahon, et al. (2003) Curr. Topics Devel. Biol. 53:1-114). Thus, the many aspects of Shh signaling were analyzed and it was found that cadmium-exposure did not perturb Shh transcription or translation; however, Shh signaling was decreased as reflected by lowered polarizing activity and expression of a luciferase reporter under control of a promoter containing multiple Gli binding sites (Sasaki, et al. (1997) Development 124:1313-1322). These data indicate that cadmium exposure modulates Shh activity in the cells where it is produced, not in Shh receiving cells. Not to be bound by theory, it is believed that cadmium has its effect on Shh by modulating one or more post-translational processing events of Shh. Such events include post-translational cleavage of the original 45 kDa protein accompanied by carboxyterminal cholesterol modification of the active 19 kDa fragment (Porter, et al. (1995) Nature 374:363-365); palmitoylation of an N-terminal cysteine (Pepinsky, et al. (1998) J. Biol. Chem. 272:14037-14045), possibly carried out during transit in the endoplasmic reticulum; export of the dually lipid modified Shh via the action of Dispatched, a protein with 12 transmembrane domains that is required for Shh to leave the cell in which it is made (Burke, et al. (1999) Cell 99:803-815); and multimerization (Zeng, et al. (2001) supra). Any of these processes may be affected by exposure to cadmium. As Dispatched is a transmembrane protein belonging to the RND permease family of transporters, it is a likely target. Members of this family in bacteria act as proton ionophores to rid themselves of xenobiotics (antibiotic resistance) including heavy metals such as cadmium (Tseng, et al. (1999) J. Mol. Microbiol. Biotechnol. 1(1):107-25). For example, acetazolamide could interfere with the proton antiporter activity of Dispatched by changing pH in the embryo (Schreiner, et al. (1995) Teratology 52:160-168). Cadmium could act competitively with Shh to utilize a limited supply of Dispatched.

As Shh is an important signaling molecule in numerous embryonic tissues and organs and the Shh signaling pathway has been found to be involved in tumor cell growth and viability, it was determined whether Shh activity itself was essential to tumor growth. The data provided in Table 1 demonstrate that cadmium inhibited the proliferation of Shh-dependent pancreatic cancer cell line L3.6pl more effectively than cyclopamine and acetazolamide and the inhibition was dose-dependent.

TABLE 1

| Inhibitor | | | |
|---|---|---|---|
| Concentration | Cell Growth (normalized) | | |
| (μM) | Cadmium Sulfate | Acetazolamide | Cyclopamine |
| 0 | 1.0000 | 1.0000 | 1.0000 |
| 1 | 1.0341 | ND | ND |
| 10 | 0.9007 | 1.0312 | 0.7509 |
| 30 | 0.7200 | ND | ND |
| 100 | 0.0041 | 1.0370 | ND |
| 1000 | 0.0029 | 1.0130 | ND |

Cell growth in inhibitor was normalized to cell growth in carrier. Carriers were as follows: cadmium, phosphate buffered saline; acetazolamide, DMSO; cyclopamine, ethanol.
ND, not determined.

Therefore, these data indicate that the inhibition of Shh activity itself by cadmium is an effective means of reducing the growth of tumors dependent upon Shh. This novel approach is in contrast to previously described use of antagonists (e.g., cyclopamine and SANTI1-4) directed against proteins located downstream of the Shh receptor in the Shh signal transduction pathway.

Accordingly, an agent (e.g., cadmium) which inhibits Shh activity is useful for reducing the proliferation of a cancer cell which is dependent upon Shh activity. Thus, the present invention provides a method for reducing cancer cell proliferation by contacting a cancer cell with an effective amount of an agent which inhibits the activity of Shh thereby reducing the proliferation of the cancer cell. Means for monitoring the reduction of cancer cell proliferation are disclosed herein. In one embodiment, the agent is cadmium.

Further, an agent (e.g., cadmium) which inhibits Shh activity is used therapeutically or prophylactically for preventing or treating a cancer which is dependent upon Shh activity. Such a method involves administering an effective amount of an agent which inhibits the activity of Shh thereby preventing or treating the cancer. In one embodiment, the agent is cadmium.

In general, an effective amount is considered an amount that decreases or inhibits cancer cell proliferation such that tumor development is arrested and/or tumor size is reduced. Desirably, the agent causes a 10%, 20%, 30%, 40%, 50%, 60%, 70%, 80%, 90%, 95%, 98%, or 100% decrease in cancer cell proliferation or tumor size when compared to otherwise same conditions wherein the agent is not present.

Individuals who can benefit from such treatment include individuals having or at risk of having a cancer such as colorectal, endometrial, gastric, hepatocellular, hepatoblastoma, kidney, medulloblastoma, melanoma, ovarian, pancreatic tumors, pilomatricoma, prostate, thyroid, uterine, and the like. Individuals having cancer generally refer to patients who have been diagnosed with cancer, whereas individuals at risk of having cancer may have a family history of cancer or exhibit one or more signs or symptoms associated with cancer (e.g., a tumor, increased pain perception, weakness).

It is contemplated that agents such as cadmium can also be administered therapeutically (including prophylactically) in other diseases or disorders involving increased (relative to normal, or desired) levels of Shh function, for example, where any member of the Shh signaling pathway; and in diseases or disorders wherein in vitro (or in vivo) assays indicate the utility of Shh antagonist administration. In vitro assays with cells of patient tissue sample or the appropriate cell line or cell type, to determine therapeutic utility, can be carried out as described herein.

For therapeutic use, the agent (e.g., cadmium), or a pharmaceutically acceptable salt thereof, is generally formulated with a pharmaceutically acceptable vehicle, such as water, buffered saline, polyol (for example, glycerol, propylene glycol, liquid polyethylene glycol and the like) or suitable mixtures thereof. The optimum concentration of the active ingredient(s) in the chosen vehicle can be determined empirically, according to procedures well-known to medicinal chemists. As used herein, pharmaceutically acceptable vehicle includes any solvent, dispersion medium, and the like which may be appropriate for the desired route of administration of the pharmaceutical preparation. The use of such vehicle for pharmaceutically active substances is known in the art. Suitable vehicles and their formulation inclusive of other proteins are described, for example, in Remington: The Science and Practice of Pharmacy, Alfonso R. Gennaro, editor, 20th ed. Lippingcott Williams & Wilkins: Philadelphia, Pa., 2000.

A pharmaceutical composition containing an agent (e.g., cadmium) which inhibits Shh activity and a pharmaceutically acceptable vehicle can be used alone or in combination with other well-established agents useful for preventing or treating cancer. Whether delivered alone or in combination with other agents, the pharmaceutical composition of the present invention can be delivered via various routes and to various sites in a mammalian, particularly human, body to achieve a particular effect. One skilled in the art will recognize that, although more than one route can be used for administration, a particular route can provide a more immediate and more effective reaction than another route. For example, intradermal delivery may be advantageously used over inhalation for the treatment of melanoma. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

Those of ordinary skill in the art can readily optimize effective doses and co-administration regimens as determined by good medical practice and the clinical condition of the individual patient. Regardless of the manner of administration, it can be appreciated that the actual preferred amounts of active agent in a specific case will vary according to the particular formulation and the route of administration. The specific dose for a particular patient depends on age, body weight, general state of health, on diet, on the timing and route of administration, on the rate of excretion, and on medicaments used in combination and the severity of the particular disorder to which the therapy is applied. Dosages for a given subject can be determined using conventional considerations, e.g., by customary comparison of the differential activities of the selected agent and of a known agent, such as by means of an appropriate conventional pharmacological protocol. Furthermore, chelating agents can be used as appropriate to modulate the toxicity of agents such as cadmium.

In a manner very similar to cadmium, thalidomide causes dysmorphogenesis of limbs (Shum, et al. (2003) Birth Defects Res. Part C Embryo. Today 69(2):102-22). Thus, it is contemplated that thalidomide and analogues thereof (e.g., CC-5013; Tohnya, et al. (2004) J. Chromatogr. B Analyt. Technol. Biomed. Life Sci. 811(2):135-141) may also be inhibiting the activity of Shh and be useful in the inhibition of cancer cell proliferation and cancer prevention or treatment.

Having demonstrated Shh activity as a target for inhibiting the proliferation of a cancer cell, the present invention further provides a method for identifying an agent which inhibits or reduces the activity of the Shh receptor in a cell and methods for using said agent to reduce cancer cell proliferation thereby preventing or treating cancer. The screening method involves contacting a cell expressing Shh with a test agent and determining whether the test agent inhibits or reduces the activity of Shh in the cell as compared to the cell not contacted with the test agent. As used herein, Sonic Hedgehog or Shh for use in the method of the invention refers to Shh proteins, or functional equivalents thereof, from any species. A wide variety of Shh proteins from a number of species are known, including, for example, human (Accession No. EAL23913), chicken (Accession No. NP_{—}990152), mouse (Accession No. AAH63087), zebrafish (Accession No. A49426), Drosophila (Accession No. AAA28604), etc. A cell expressing Shh can be any cell including a prokaryotic cell, a yeast cell, or a cell isolated from a plant, insect, worm, frog, fly, fish, mouse, rat, monkey, animal or mammal. Shh can be exogenously expressed or be recombinantly expressed in the cell in accordance with standard recombinant technology practices. Desirably, the cell has a naturally occurring Shh signal transduction pathway. If the cell does not have a Shh signaling pathway, the cell can be transfected with sequences encoding for the members of the Shh signaling pathway necessary for achieving the method of the invention. In general, the cell is capable of being propagated in vitro. Particularly suitable cells for carrying out the screening method of the present invention include Shh-Light 2 cells or cancer cells which are dependent upon Shh activity.

In the screening method of the invention, a cell expressing Shh is contacted with a test agent and the activity of Shh is determined, for example, by measuring the expression of a luciferase reporter under control of a promoter containing multiple Gli binding sites (Sasaki, et al. (1997) supra). The activity of Shh in a cell contacted with the test agent is then compared to the activity of Shh in a cell in the absence of the test agent so as to be able to determine the effects of the test agent on Shh activity and therefore Shh-mediated cellular proliferation.

In general, it is desirable that the assay of the invention is amenable to automated, cost-effective high throughput screening. As such, the test agent can be a member of a library of natural or synthetic compounds (e.g., a combinatorial library). The test agent can be any type of molecule which can affect Shh activity, including, for example, polypeptides, antibodies, nucleic acids, carbohydrates, small organic molecules, inorganic molecules, and the like.

A variety of techniques are available in the art for generating synthetic combinatorial libraries of small organic molecules. See, for example, Blondelle et al. (1995) Trends Anal. Chem. 14:83; Dooley, et al. (1993) Proc. Natl. Acad. Sci. U.S.A. 90:10811-10815; U.S. Pat. No. 5,359,115; U.S. Pat. No. 5,362,899; U.S. Pat. No. 5,288,514; WO 94/08051; U.S. Pat. No. 5,736,412; U.S. Pat. No. 5,712,171; Chen, et al. (1994) JACS 116:2661; Kerr, et al. (1993) JACS 115:252; WO 92/10092; WO 93/09668; WO 91/07087; and WO 93/20242).

In yet another embodiment, test agents which can be screened within the scope of the present invention include antibodies and fragments containing the binding domain thereof, directed against Shh. Accordingly, Shh proteins, fragments or analogs or derivatives thereof, in particular, human Shh proteins or fragments thereof, can be used as immunogens to generate anti-Shh protein antibodies. Such antibodies can be polyclonal, monoclonal, chimeric, single chain, Fab fragments, or from a Fab expression library.

Various procedures known in the art can be used for the production of polyclonal antibodies to a Shh protein or peptide. For the production of antibody, various host animals can be immunized by injection with the native Shh protein, or a synthetic version, or fragment thereof, including but not limited to rabbits, mice, rats, etc. Various adjuvants can be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhold limpet hemocyanins, dinitrophenol, and potentially useful human adjuvants such as BCG (bacille Calmette-Guerin) and Corynebacterium parvum.

For preparation of monoclonal antibodies directed toward a Shh protein sequence, any technique which provides for the production of antibody molecules can be used. For example, the hybridoma technique originally developed by Kohler and Milstein ((1975) Nature 256:495-497), as well as the trioma technique, the human B-cell hybridoma technique (Kozbor, et al. (1983) Immunology Today 4:72), and the EBV-hybridoma technique to produce human monoclonal antibodies (Cole, et al. (1985) in: Monoclonal Antibodies and Cancer Therapy, Alan R. Liss, Inc., pp. 77-96).

Test agents prepared by any of the above techniques can then be tested in an assay as described herein, e.g., in a high-throughput assay, to measure their activity towards modulation of Shh activity. Repeated iterations of synthesis and testing can be used as part of a medicinal chemistry program to identify compounds which have increased activity, reduced side-effects, etc.

After identifying certain test agents in the subject assay, the efficacy and specificity of the selected agent can be tested both in vitro and in vivo. Whether for subsequent in vivo testing, or for administration to an animal as an approved drug, agents identified in the subject assay can be formulated in pharmaceutical preparations for in vivo administration to an animal such as a human.

In certain embodiments of the assay method of the present invention, it will be desirable to monitor the growth state of cells in the culture, e.g., cell proliferation, differentiation and/or cell death. Methods of measuring cell proliferation are well-known in the art and most commonly include determining DNA synthesis characteristic of cell replication. There are numerous methods in the art for measuring DNA synthesis, any of which can be used according to the invention. For example, DNA synthesis can be determined using a radioactive label (³H-thymidine) or a labeled nucleotide analogue (BrdU) for detection by immunofluorescence.

In vitro assays which can be used to determine whether administration of a specific agent will be therapeutically useful, include in vitro cell culture assays in which a patient tissue sample is grown in culture, and exposed to or otherwise administered the agent, and the effect of such agent upon the tissue sample is observed. In one embodiment, a sample of cells is taken from a patient having or suspected of having a malignancy, wherein the malignant cells are plated out or grown in culture, and the cells are then exposed to the agent. An agent which inhibits survival or growth of the malignant cells is selected for therapeutic use in vivo. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, etc.

In in vitro assays wherein the sample of cells is from a patient having a malignancy, such a malignancy can include certain cancers such as colorectal, endometrial, gastric, hepatocellular, hepatoblastoma, kidney, medulloblastoma, melanoma, ovarian, pancreatic tumors, pilomatricoma, prostate, thyroid, uterine, etc. but are not limited to those described herein.

In in vitro assays wherein the sample of cells is from a patient suspected of having a malignancy (i.e., pre-neoplastic), the cell are similarly plated out or grown in vitro, and exposed to a test agent. An agent which results in a cell phenotype that is more normal (i.e., less representative of a pre-neoplastic state, neoplastic state, malignant state, or transformed phenotype) is selected for therapeutic use. Many assays standard in the art can be used to assess whether a pre-neoplastic state, neoplastic state, or a transformed or malignant phenotype, is present. For example, characteristics associated with a transformed phenotype (a set of in vitro characteristics associated with a tumorigenic ability in vivo) include a more rounded cell morphology, looser substratum attachment, loss of contact inhibition, loss of anchorage dependence, release of proteases such as plasminogen activator, increased sugar transport, decreased serum requirement, expression of fetal antigens, disappearance of the 250,000 dalton surface protein, etc. (see, Luria, et al. (1978) General Virology, 3d Ed., John Wiley & Sons, New York pp. 436-446).

In other embodiments, the in vitro assays described supra can be carried out using a cell line, rather than a cell sample derived from the specific patient to be treated, in which the cell line is derived from or displays one or more characteristic associated with the malignant, neoplastic or pre-neoplastic disorder desired to be treated or prevented.

The invention is described in greater detail by the following non-limiting examples.

### EXAMPLE 1

### Materials and Methods

Cadmium-induced Forelimb Ectrodactyly. C57BL/6CrIBR mice were purchased from Charles River Laboratories (Wilmington, Mass.). Individual males were placed in female cages for the last 2-3 hours of the 12 hour dark cycle (10:00 pm to 10:00 am). The presence of a vaginal plug indicated a successful mating and 9:00 am was considered time zero of pregnancy. On day 7 of gestation, plugged females were lightly anesthetized with Isofluorane and checked for pregnancy by abdominal palpation. The pregnant females were administered CdSO₄ (SIGMA, St. Louis, Mo.), 4 mg/kg in phosphate-buffered saline (PBS), intraperitoneally, at 9:00 pm on day 9. These pregnant females were then allowed free access to food and water until day 18 when they were sacrificed by Isofluorane overdose. The fetuses were removed from the uterus and prepared for skeletal analysis by double-staining of cartilage and bone (Kuczuk and Scott (1984) supra).

Polarizing Activity. Fertilized White Leghorn eggs (SPAFAS) were incubated at 38° C. On the second day, 1 to 2 mL of albumin were removed so that the eggs could be windowed the next day without damaging the embryo. After windowing, 3 drops of penicillin/streptomycin (50 units/mL) were added and the window closed with SCOTCH™ tape. On the fourth day of incubation a piece of day 10.5 mouse embryo right forelimb was excised from the posterior aspect (i.e., ZPA) of cadmium sulfate- or PBS-exposed embryos and grafted between the ectoderm and mesenchyme at the anterior margin of a stage 19/20 (Hamburger and Hamilton (1951) J. Morphol. 88:49-92) chick right wing bud. After grafting, 3 drops of penicillin/streptomycin were added, the window closed with tape and the egg replaced in the incubator for 7 additional days. At this time the chick embryo was sacrificed and the right wing bud processed for skeletal examination (Kuczuk and Scott (1984) supra). After staining, the digit pattern was determined and the index of polarizing activity was assessed according to standard methods (Honig, et al. (1981) J. Embryol. Exp. Morph. 62:203-216),

Shh Signaling Activity. Pregnant C57BL/6 mice were treated with CdSo₄, 4 mg/kg, or PBS, 10 mL/kg, at 9:00 pm on day 9 of gestation. Twenty-four hours post-treatment the dams were sacrificed. The implantation sites were removed and frozen in liquid nitrogen. Samples were stored at −80° C. until use in the Shh activity assay.

To create a sample of cell lysate, a litter of implantation sites was thawed and the embryos dissected free of their surrounding membranes. Somites were counted and the embryo limb staged according to standard practices (Wanek, et al. (1989) J. Exp. Zool. 249:41-49). Right forelimbs (stages 3 to 3.5) were pooled in an EPPENDORF® tube, the excess dissection fluid removed and replaced with 200 μL serum-free Dulbecco's Modified Eagle Medium (DMEM). Samples were sonicated and DNA measured using the HOEFER™ DYNA QUANT™ 200 Fluorimeter. After DNA quantification, fetal bovine serum was added so that the final concentration of serum was 0.5%.

Assay of SHH signaling activity was conducted in Shh-Light 2 cells, an NIH-3T3-derived stable cell line containing an integrated Gli-luciferase and constitutive Renilla luciferase reporters. The reliability of this assay system to monitor Shh signaling is well-established in the art (Taipale, et al. (2000) Nature 406:1005-1009). Shh-Light 2 cells were cultured in DMEM containing 10% fetal bovine serum in a 96-well plate (2.5×10⁵ cells/well) for approximately 24 hours (confluence). The media was removed and replaced by cell lysate containing 50 or 25 ng/mL of DNA in 100 mL of DMEM (0.5% FBS) added to the wells in duplicate. A duplicate set of wells contained only 100 mL of DMEM (0.5% FBS) to serve as background. Cells were incubated for 24 hours. Using the DUAL-LUCIFERASE® Reporter Assay System (PROMEGA®, Madison, Wis.), luciferase activity was measured and normalized to a Renilla control.

Whole Mount In Situ Hybridization. Assays were performed in accordance with standard methods (Wilkinson and Nieto (1993) Meth. Enzymol. 225:361-373). Embryos from control and cadmium-exposed pregnancies were processed together in the same vessel, examined and photographed using an OLYMPUS® SZX9 stereomicroscope. Digoxigenin-labeled riboprobes were transcribed from linearized DNA templates.

Real Time Reverse Transcribed-Polymerase Chain Reaction (RT-PCR). Precise levels of message expression of Shh, Ptch1, and Gli3 were performed by RT-PCR. The STRATAGENE® nanoprep RNA kit (STRATAGENE®, La Jolla, Calif.) was used to isolate total RNA from forelimb buds of day 10.5 embryos 24 hours after cadmium sulfate or PBS administration. Right and left forelimb buds from a single litter (n=3) were pooled separately and equal amounts of total RNA were reverse-transcribed using SUPERSCRIPT™ II (INVITROGEN™, Carlsbad, Calif.) in the presence of oligo-dT or random primers. For quantitative PCR, aliquots of the reverse transcription reaction were amplified in the presence of SYBR® Green (Quantitect SYBR® green PCR kit; QIAGEN®, Valencia, Calif.) and gene specific primers for Shh, Ptch1, Gli3 and aTub. Chip array data and preliminary experiments indicated that α-tubulin mRNA levels were unchanged by cadmium exposure and thus was used to normalize the quantitative PCR data. The PCR amplifications and monitoring of product generation per cycle were performed using a DNA Engine OPTICON® 2 Continuous Fluorescence Detector and evaluated using its software. Gene expression levels were determined by gene-specific standard curves in the linear amplification phase. All values were normalized to tubulin within the same sample.

Western Analysis. Forelimb buds from E10.5 vehicle or cadmium-exposed embryos were excised, cut in half, and the posterior half collected for western blot analysis. Pooled limb bud samples were lysed in RIPA buffer in the presence of a protease inhibitor cocktail and then homogenized by passage through a narrow gauge needle followed by water bath sonication. Samples were centrifuged at 2000 g for 5 minutes. Laemmli buffer (5×) was then added to the samples and boiled for 10 minutes. Samples were then separated on SDS-PAGE gels and transferred to nitrocellulose. After probing the membranes for Shh with mAB5E1 (Developmental Studies Hybridoma Bank, Iowa City, Iowa) the membranes were stripped and probed using a monoclonal antibody to tubulin β-tubulin mouse monoclonal, TUB 2.1; SIGMA, St. Louis, Mo.).

### EXAMPLE 2

### Cadmium-Induced Postaxial Forelimb Ectrodactyly

CdSo₄, 4 mg/kg, was administered intraperitoneally on day 9 of gestation. This dosage was chosen because it induced little or no increase of embryo lethality (a 20% rate of resorption occurs with the C57Bl/6 mice), and because this dosage induced a high frequency of postaxial forelimb ectrodactyly, 85% (Table 1).

TABLE 1

| | | | No. Fetuses with |
|---|---|---|---|
| No. | No. Implantation | No. Resorptions | Postaxial Forelimb |
| Dams | Sites | (%) | Ectrodactyly (%) |
| 8 | 68 | 15 (22) | 45 (85) |

The most common phenotype induced by this dosage was loss of digit five (5) from both forelimbs, but right-sided predominance (Kuczuk and Scott (1984) supra; Hovland, et al. (1999) supra; Layton and Layton (1979) supra; Messerle and Webster (1984) supra) was clearly evident (Table 2).

TABLE 2

| | | Bilateral | | |
|---|---|---|---|---|
| RFL Only | LFL Only | R > L | R = L | R < L |
| 12* | 4 | 5 | 21 | 3 |

*Number of fetuses.

Subsequent study of polarizing activity and Shh signaling activity focused on the right forelimb, which was affected by postaxial ectrodactyly in 78% of the fetuses.

The capability of the mouse embryo ZPA to induce limb reduplication was severely inhibited by exposure to CdSO₄. The ZPA from the forelimb of a day 10.5 mouse embryo has been shown to induce a polarizing “score” ranging from 60-80 (Bell, et al. (1999) Am. J. Physiol. 276:C788-C795), an outcome observed herein (Table 3).

TABLE 3
Induced Digit Pattern (Score)*

| PBS | CdSo₄ |
|---|---|
| 23334 | 234 |
| 23234 | 234 |
| 23334 | 234 |
| 23334 | 234 |
| 43x234 | x234 |
| (73) | x234 |
| | x234 |
| | x234 |
| | 22234 |
| | (11) |

Italics indicates constitutive digits.
*x = unidentifiable partial duplicated digit; scores as an individual 0.5 if most posterior.
Score derived by points assigned to most posterior duplicated digit; digit 4 = 3, digit 3 = 2, digit 2 = 1, no additional digit = 0; sum of scores/sum of highest possible score × 100 = (score) (Honig, et al. (1981) supra).

When the grafted ZPA tissue was from a cadmium-exposed embryo, this “score” was decreased to 11 (Table 3). Of the nine grafted ZPAs from cadmium-exposed embryos only one was able to induce a recognizable reduplicated digit. Four other grafts induced a minor cartilaginous structure unrecognizable as a digit. The remaining four grafts were unable to induce any sign of digit reduplication. These data indicated that polarizing activity, an indicator of A/P patterning activity and of Shh signaling, was drastically down-regulated to about 15% of normal by exposure to cadmium.

A more direct and specific assay of Shh signaling is provided in Shh-Light 2 cells. These NIH-3T3 cells are stably transfected with a luciferase reporter under control of a promoter containing multiple Gli binding sites (Sasaki, et al. (1997) supra). Culture of these cells with a medium or cellular homogenate containing Shh activates reporter expression and that activity can be quantitated luminometrically. The activity is antagonized by an antibody to Shh, 5E1, and by cyclopamine, an inhibitor of Shh signaling (Taipale, et al. (2000) supra; Zeng, et al. (2001) Nature 411:719-720). It was found that a lysate of E10.5 right forelimb bud cells from a cadmium-exposed embryo activated Shh signaling only to a level of 25% when compared to the lysate from control embryo right forelimb buds.

The expression pattern of four genes (i.e., Shh, Ptch1, Gli3, and Fgf8) in the right forelimb of control and cadmium-exposed embryos was examined by whole mount in situ hybridization 24 hours after treatment. The expression of Shh was restricted to the posterior mesenchymal cells of the developing murine limb bud. No discernable difference was observed in location or intensity of Shh expression in embryos exposed to cadmium.

Ptch1 transcribes a transmembrane protein that acts as a receptor for Shh. The transcription of this gene is upregulated by Shh and thus can be used as an indicator of Shh activity; thus Ptch1 is normally expressed at high intensity posteriorly where Shh is expressed and trails off anteriorly as the distance from Shh expression increases. This pattern of Ptch1 expression was seen in the control embryo right forelimb, and the pattern was unchanged by cadmium exposure.

Gli3 is a zinc finger transcriptional regulator whose activity to stimulate or repress downstream targets, is controlled by Shh signaling (Wang, et al. (2000) Cell 100:423-434). Gli3 expression begins during very early stages of limb morphogenesis when it is expressed throughout most of the limb, except at the extreme posterior (Buscher and Ruther (1998) Dev. Dyn. 211:88-96). This area of posterior non-expression expands as the limb bud progresses developmentally so that by the stage observed herein, expression is generally restricted to the anterior limb bud mesenchyme. Exposure to cadmium did not alter Gli3 expression in a manner that could be discerned by whole mount in situ hybridization.

The outgrowth of the vertebrate limb along the proximal/distal axis is facilitated/controlled by the apical ectodermal ridge (AER). This function of the AER is carried out by members of the Fgf gene family especially Fgf8 (Martin (1998) Genes and Development 12:1571-1586). Fgf8 is expressed in the early ventral limb ectodermal precursors of the AER (Loomis, et al. (1998) Development 125:1137-1148; Bell, et al. (1998) Mech. Dev. 74:41-50) and throughout the mature AER. At day 10.5, 24 hours after cadmium administration, there was a slight truncation of Fgf8 staining posteriorly. It is contemplated that this loss of AER function posteriorly could underlie the subsequent loss of posterior digits.

In addition to analyzing gene expression patterns, quantitative evaluation of mRNA was determined. To evaluate potential modest alterations of expression of Shh and downstream targets, quantitative RT-PCR was used to measure Shh, Ptch1 and Gli3. A reduction of mRNA levels was not observed for these three genes in the limb buds of cadmium-exposed mouse embryos thereby substantiating the visual results from the whole mount in situ hybridization studies.

To ascertain whether cadmium was lowering Shh signaling by impeding the translation of Shh mRNA into protein, the content of Shh in the posterior half of E10.5 forelimb buds (24 hours after cadmium sulfate administration) was determined by western blot analysis. It was found that limb buds from cadmium-exposed embryos had a similar content of Shh as PBS-exposed controls.

What is claimed is:

1. A method for identifying an agent which inhibits the activity of Sonic Hedgehog in a cell comprising contacting a cell expressing Sonic Hedgehog with a test agent and determining whether the test agent inhibits the activity of Sonic Hedgehog in the cell as compared to a cell not contacted with the test agent.

2. A method for reducing the proliferation of a cancer cell comprising contacting a cancer cell which is dependent upon Sonic Hedgehog activity with an effective amount of an agent which inhibits the activity of Sonic Hedgehog thereby reducing the proliferation of the cancer cell.

3. The method of claim 2, wherein the agent is cadmium.

4. A method for preventing or treating cancer comprising administering to a patient having or at risk of having a cancer which is dependent upon Sonic Hedgehog activity with an effective amount of an agent which inhibits the activity of Sonic Hedgehog thereby preventing or treating the cancer.

5. The method of claim 4, wherein the agent is cadmium.
